(19) United States
(12) Patent Application Publication
(10) Pub. No.: US 2006/0172296 A1
(43) Pub. Date: Aug. 3, 2006

(54) Novel gene associated with rheumatoid arthritis

(76) Inventors: Masahiro Takeuchi, Tsukuba-shi (JP); Noboru Yamaji, Tsukuba-shi (JP); Jun Takasaki, Tsukuba-shi (JP); Masahiko Akamatsu, Tsukuba-shi (JP); Kazuhisa Tsunoyama, Tsukuba-shi (JP); Masayoshi Harigai, Itabashi-ku (JP)

Correspondence Address: SUGHRUE MION, PLLC, 2100 PENNSYLVANIA AVENUE, N.W., SUITE 800, WASHINGTON, DC, 20037, US

(21) Appl. No.: 10/521,940

(22) PCT Filed: Jul. 18, 2003
(86) PCT No.: PCT/JP03/09180

§ 371 Date: Jan. 21, 2005

(30) Foreign Application Priority Data
Jul. 22, 2002 (JP) 2002-211951

(51) Int. Cl. C12Q 1/68; C07H 21/04; C12P 21/06; C07K 14/54

(52) U.S. Cl. 435/6; 435/69.1; 435/320.1; 435/325; 530/351; 536/23.5

(57) ABSTRACT

It is disclosed that novel polypeptides which has enhanced expression levels in the synovial tissues of human chronic rheumatoid arthritis (RA) patients compared with the synovial tissues of human osteoarthritis (OA) patients, polynucleotides encoding the polypeptides, vectors comprising the polynucleotides, transformant cells comprising the vectors and an antibody against the polypeptide. It is also disclosed that polynucleotides specifically hybridizing with the polynucleotides and having at least 15 nucleotides, a detection method which is useful for RA diagnosis, and a detection kit which is useful for RA diagnosis.

### TECHNICAL FIELD

The present invention relates to a novel polypeptide relating to chronic rheumatoid arthritis (RA), a polynucleotide encoding the polypeptide, vectors comprising the polynucleotide, a transformant cell comprising the vector and an useful method for detecting RA diagnosis.

### BACKGROUND OF THE INVENTION

RA is a chronic inflammatory disease of unknown origin, which has the mainlocus of lesion in the synovial tissue and causes flare, swelling, heat sensation, pain, movement restriction and destruction of joints. Overproduction of inflammatory cytokines such as interleukin-1 (IL-1), interleukin-6 (IL-6), interleukin-8 (IL-8), interleukin-12 (IL-12), interleukin-15 (IL-15), interleukin-18 (IL-18), tumor necrosis factor-α (TNF-α) and the like, nitric oxide (NO), prostaglandins (PGs) and the like is known in the synovial tissue of RA (cf. non-patent reference 1). Additionally, immunocytes which constitute the synovial tissue activate each other through molecules on cellular surface, for example CD40/CD40 ligand systems and ICAM-1 (intercellular adhesion molecule-1)/LFA-1 (leukocyte adhesion molecule-1) systems and relate to the prolongation of inflammatory reaction (see non-patent reference 2). In recent years, a therapeutic method aimed at IL-1, IL-6 or TNF-α has been developed using a monoclonal antibody and a soluble receptor, and its efficacy is drawing attention (cf. non-patent reference 3). However, there is a group of patients in which complete remission cannot be introduced by the conventional therapeutic method which uses a therapeutic target molecule as the mechanism (cf. non-patent reference 4). Accordingly, identification of a new therapeutic target molecule which is different from the already known reports is expected.

According to the progress of the analysis of the pathology of RA, the meaning of RA diagnosis at early stage and the meaning of its early therapy have been drawing attention, together with a challenge of a therapeutic method for suppressing of excessive synovial proliferation to suppress or prolong joint bone destruciton, using synovial inflammation and immune abnormality as targets. On the other hand, a certain university in USA proposes a criterion concerning RA classification (see non-patent reference 5). Since the criterion is a simple standard and RA includes highly various pathologic patterns, RA diagnosis, especially quantitative and simple diagnosis of RA has been considered to be much difficult. Accordingly, a quantitative and simple method for diagnosis of RA has been strongly desired.

The sequence which relates to the sequence of RA2 as one of the polypeptides of the present invention is registered as AK094461 (2002. 7. 15) on the database genpept and is described in the patent reference 1, the patent reference 2 and the patent reference 3. In the patent reference 1, there is a description that an antagonist against the polypeptide can be used for the treatment for reducing the inflammatory reaction and can be administered for treating for example cardiac diseases with arteriosclerosis, inflammatory colon disease, Crohn's disease, chronic rheumatoid arthritis and pancreatitis. The patent reference 2 describes a great number of proteins comprising sequences homologous with that of RA2 and these proteins are useful for the treatment of cancer, digestive disorders, immune disorders, endocrine diseases (for example, diabetes mellitus), neurological diseases (for example, Alzheimer disease, Parkinson's disease, Creutzfeldt-Jacob disease, encephalomyelitis, menigitis, and schizophrenia) and connective tissue diseases (for example, osteoporosis and arthritis). In the patent reference 3, there is a description of a great number of genes comprising sequences homologous with the sequence of the RA2 gene and a description that on the basis of the distributions of individual expressions in tissues that these individual genes are useful for the treatment of proliferative diseases, cancer, tumor, hematopoietic diseases, immune diseases, AIDS, autoimmune diseases (for example, chronic rheumatoid arthritis), inflammation, allergy, nerological diseases (for example, Alzheimer disease), dysgnosia, schizophrenia, asthma, dermatoses (for example, psoriasis), sepsis, diabetes, artheriosclerosis, cardiovascular disorders, kidney disorders, gastrointestinal diseases, disorders related to pregnancy, endocrine disorders and infectious diseases.

A sequence homologous with the sequences of some of the polypeptides of the present invention, namely DGPP1L and DGPP1S are described in the database genpept as BC033025 (2002. 6. 24).

In the patent reference 4, the patent reference 5 and the patent reference 6, sequences homologous with DGPP2L which is one of the polypeptides of the present invention are described. Among them, in Abstract in the patent reference 4, it is described that the sequence homologous with-DGPP2L is useful for the diagnosis and treatment of malignant tumor, blood diseases, HIV infection, immune diseases and various inflammations. In the patent reference 5, it is described that the sequence homologous with DGPP2L is useful for the diagnosis, treatment, preveniton and prognosis of diseases including tumor diseases such as per-proliferative diseases; that the sequence is also useful for the treatment of neurological diseases, immune diseases, muscular diseases, genital diseases, gastrointestinal diseases, lung diseases, circulatory diseases, or kidney diseases; and that the sequence is additionally useful as an agent for detecting diseases related to the abnormal expression and activity of the polypeptide and is useful for the treatment of cancer and chronic rheumatoid arthritis. In the patent reference 6, a sequence highly homologous with DGPP2L is disclosed and it is described that the sequence is useful for the detection, treatment, prevention or prognosis of per-proliferative diseases (for example, cancer), immunodeficiency diseases (for example, AIDS), autoimmune diseases (for example, arthritis), neurological diseases (for example, Alzheimer disease), metabolic disorders (for example, phenylketonuria), inflammatory diseases (for example, asthma), circulatory diseases (for example, artheriosclerosis), blood diseases (hemophilia), genital diseases (for example, infertility) and infectious diseases (for example, influenza).

In the patent reference 4, it is described that the same sequence with DGPP2S which is one of the polypeptides of the present invention, while sequences homologous with the sequence are describes in the patent reference 5 and the patent reference 6.

(Patent reference 1) Pamphlet of International Publication No. 00/42070

(Patent reference 2) Pamphlet of International Publication No. 01/77137

(Patent reference 3) Pamphlet of International Publication No. 01/32910

(Patent reference 4) Pamphlet of International Publication No. 02/26798

(Patent reference 5) Pamphlet of International Publication No. 01/55163

(Patent reference 6) Pamphlet of International Publication No. 01/55301

(Non-patent reference 1) “The Journal of Experimental Medicine”, (USA), 1991, Vol.173, p. 569-574

(Non-patent reference 2) “The Journal of Rheumatology”, (Canada), 2002, Vol.29, p. 875-882

(Non-patent reference 3) “Current Pharmaceutical Biotechnology”, (USA), 2000, Vol. 1, p. 217-233

(Non-patent reference 4) “Nature Reviews Immunology”, (UK), 2002, Vol. 2, p. 364-371

(Non-patent reference 5) “Medicine”, J. Axford, ed., (USA), Blackwell Science, 1996, p. 3.18-3.22

### DISCLOSURE OF THE INVENTION

As the result of various investigations, the inventors of the present invention successfully obtained six full-length gene sequences, using cDNA derived from human spleen as a template (Example 1). Five of them were novel genes. Additionally, the inventors of the present invention successfully allowed the expression of proteins encoded by the six genes in animal cell strains (Example 2). Further, the inventors of the present invention found that the expression levels of the six genes were elevated in the synovial tissues of human RA patients. Still further, the inventors of the present invention found that the expression level of each of the genes was elevated in accordance with the score of pathologic RA findings, i.e. that the severity of the inflammatory symptoms of RA is related with the elevation of the expression level of each of the genes (Example 4). Based on these findings, a method which is useful as a method for RA diagnosis by which progress of RA is detected was enabled.

From these results, the inventors of the present invention provided a novel gene, a protein encoded by the genes, expression vectors harboring the gene, a cell transformed with the expression vector or an antibody, and a detection method useful for RA diagnosis and a detecting kit therefor. Thus, the present invention has been achieved.

Specifically, the present invention relates to the following:

[1] (1) A polypeptide comprising an amino acid sequence represented by SEQ ID NO:2, SEQ ID NO:4, SEQ ID NO:6, SEQ ID NO:8 or SEQ ID NO:10 and having enhanced expression in chronic rheumatoid arthritis patients or

(2) a polypeptide comprising an amino acid sequence represented by SEQ ID NO:2, SEQ ID NO:4, SEQ ID NO:6, SEQ ID NO:8 or SEQ ID NO:10 in which 1 to 10 amino acids are substituted, deleted and/or inserted and having enhanced expression in chronic rheumatoid arthritis patients.

[2] A polypeptide consisting of an amino acid sequence represented by SEQ ID NO:2, SEQ ID NO:4, SEQ ID NO:6, SEQ ID NO:8 or SEQ ID NO:10.

[3] A polynucleotide encoding the polypeptide according to [1] or [2].

[4] An expression vector comprising the polynucleotide according [3].

[5] A cell transformed with the expression vector according to [4].

[6] A antibody which binds to a polypeptide comprising an amino acid sequence represented by SEQ ID NO:4 and having enhanced expression in chronic rheumatoid arthritis patients, a polypeptide comprising an amino acid sequence represented by SEQ ID NO:4 in which 1 to 10 amino acids are substituted, deleted and/or inserted and having enhanced expression in chronic rheumatoid arthritis patients or a polypeptide consisting of an amino acid sequence represented by SEQ ID NO:4.

[7] A polynucleotide which specifically hybridizes to a polynucleotide represented by SEQ ID NO:1, SEQ ID NO:3, SEQ ID NO:5, SEQ ID NO:7 or SEQ ID NO:9 and has at least 15 nucleotides.

[8] A method for detecting rheumatoid arthritis comprising

1) a step of assaying the expression level of a gene in a synovial biopsy sample of a test subject, said gene comprising

i) the nucleotide sequence according to [3] or

ii) a polynucleotide sequence encoding a polypeptide comprising an amino acid sequence represented by SEQ ID NO:12 and having enhanced expression in chronic rheumatoid arthritis patients, a polypeptide comprising an amino acid sequence represented by SEQ ID NO:12 in which 1 to 10 amino acids are substituted, deleted and/or inserted and having enhanced expression in chronic rheumatoid arthritis patients, or a polypeptide consisting of an amino acid sequence represented by SEQ ID NO:12; and

(2) a step of comparing the expression level of the gene with the expression levels thereof in a synovial biopsy sample in a normal subject or a patient with non-chronic rheumatoid arthritis.

[9] A kit for detecting chronic rheumatoid arthritis comprising forward and reverse primers designed for enabling specific amplification of a gene represented by

1) the polynucleotide according to [3] or

2) a polynucleotide encoding a polypeptide comprising an amino acid sequence represented by SEQ ID NO:12 and having enhanced expression in chronic rheumatoid arthritis patients, a polypeptide comprising an amino acid sequence represented by SEQ ID NO:12 in which 1 to 10 amino acids are substituted, deleted and/or inserted and having enhanced expression in chronic rheumatoid arthritis patients, or a polypeptide consisting of an amino acid sequence represented by SEQ ID NO:12.

Although the same sequence with RA2 which is one of the polypeptides of the present invention has not yet been known, a homologous sequence is registered as AK094461 (2002. 7. 15) on the database genpept and is also described in the patent reference 1, the patent reference 2 and the patent reference 3. The patent reference 1, the patent reference 2 and the patent reference 3 cites a great number of diseases which the polypeptide relates to and includes chronic rheumatoid arthritis and arthritis among them. However, in the patent reference 1, it is described that by only the reason that the molecule is expressed in a lymph node, a spleen, a thymus, a btestis, a small intestine, a human arterial endothelial cell, a smooth muscle, a kidney, a mast cell, an eosinophilic leukocyte, a tonsil, a pancreas, a large intestine, a peripheral blood lymphocyte, a stomach, a tachea, T cells including CD4⁺ and CD8⁺ and bone marrow, the molecule is described as a cytokine and is described that antagonists against the molecule are useful for inflammatory diseases including RA. In the patent reference 2, any example which relates to the molecule is not described. In the patent reference 3, although plural molecules are described, the examples are simply described in present form. Additionally, the patent references 1 through 3 never include any description about the presence or absence of the expression of the molecule in a synovial membrane or about the expression level thereof in RA patients. Accordingly, the fact that RA2 is useful for RA diagnosis was found by the inventors of the present invention for the first time. Further, the method for detecting RA based on the finding is present invention achieved by the inventors of the present invention for the first time.

The same or highly homologous sequence with RA3 which is one of the polypeptides of the present invention has never been known. Thus, RA3 is a polypeptide found and obtained by the inventors of the present invention for the first time. The elevation of the RA expression level according to the severity of the inflammatory symptoms of RA was found by the inventors of the present invention for the first time.

The same sequences with DGPP1L and DGPP1S which are one of the polypeptides of the present invention have never been known. Although a sequence homologous with them is registered as BC033025 on the database genpept (2002. 6. 24), the function is unknown. Further, it was found by the inventors of the present invention for the first time that DGPP1L and DGPP1S are useful for detecting RA.

The same sequence with DGPP2L which is one of the polypeptides of the present invention has not yet been known. However, homologous sequences are described in the patent reference 4, patent reference 5, and patent reference 6. Among them, in Abstract in the patent reference 4, it is described that the sequence homologous with DGPP2L is useful for the diagnosis and treatment of malignant tumor, blood diseases, HIV infection, immune diseases and various inflammations. In the patent reference 5, it is disclosed that a great number of sequences including sequences homologous with DGPP2L, and these polypeptides are useful for the diagnosis, treatment, prevention and prognosis of diseases including tumor diseases such as per-proliferative diseases; that the sequences are also useful for the treatment of nerological diseases, immune diseases, muscular diseases, genital diseases, gastrointestinal-diseases, lung diseases, circulatory diseases, or kidney diseases; and that the sequences are useful as an agent for detecting diseases involved in the abnormal expression and activity of the polypeptide and are useful for the treatment of cancer and chronic rheumatoid arthritis. However, in the reference, there is no disclosure about which sequence among these many sequences can be used for which use and about examples carried out actually.

In the patent reference 6, it is disclosed that a great number of sequences including sequences highly homologous with DGPP2L and it is described that the sequences are useful for the detection, treatment, prevention or prognosis such as per-proliferative diseases (for example, cancer), immunodeficiency diseases (for example, AIDS), autoimmune diseases (for example, arthritis), neurological diseases (for example, Alzheimer disease), metabolic disorder (for example, phenylketonuria), inflammatory diseases (for example, asthma), circulatory diseases (for example, atheroma arteriosclerosis), blood diseases (hemophilia), genital diseases (for-example, infertility) and infectious diseases (for example, influenza). However, in the patent reference 6, there is neither any specific description about examples nor specific supporting evidence for these uses. Further, in the patent references 5 and 6, there is not any description of the presence or absence of the expression of a DGPP2L analogous molecule in a synovial membrane or about the expression level thereof in RA patients. Accordingly, it is the fact found by the inventors of the present invention for the first time that DGPP2L is useful for RA diagnosis. Further, the method for detecting RA based on the finding is an invention achieved by the inventors of the present invention for the first time.

In the patent reference 4 described above, the same sequence with DGPP2S which is one of the polypeptide of the present invention is described, while sequences homologous with the sequence are described in the patent reference 5 and the patent reference 6. As described above, however, there is not any description in the patent reference 5 and the patent reference 6 about the presence or absence of the expression of the molecule in a synovial membrane or about the expression level thereof in RA patients. Accordingly, it was found for the first time by the inventors of the present invention that DGPP2L is useful for RA diagnosis. Further, the method for detecting RA based on the finding is an invention achieved for the first time by the inventors of the present invention.

### BRIEF DESCRIPTION OF THE DRAWINGS

FIG. 1 shows the expressions of the individual novel proteins.

FIG. 2 shows the expression levels of the individual gene in RA synovial fibroblast-like cells in comparison with these in OA fibroblast-like cells. The vertical axis in the figure represents relative expression level.

### BEST MODE FOR CARRYING OUT THE INVENTION

The present invention is now described in detail hereinbelow.

The polypeptides of the present invention include:

(1) a polypeptide consisting of an amino acid sequence represented by SEQ ID NO:2, SEQ ID NO:4, SEQ ID NO:6, SEQ ID NO:8 or SEQ ID NO:10;

(2) a polypeptide comprising an amino acid sequence represented by SEQ ID NO:2, SEQ ID NO:4, SEQ ID NO:6, SEQ ID NO:8 or SEQ ID NO:10 and having enhanced expression specific to RA patients or a polypeptide comprising an amino acid sequence represented by SEQ ID NO:2, SEQ ID NO:4, SEQ ID NO:6,-SEQ ID NO:8 or SEQ ID NO:10 in which 1 to 10 amino acids in an amino acid and having enhanced expression specific to RA patients; (called functionally equivalent variant hereinafter); and

(3) a polypeptide comprising an amino acid sequence with 90% or more homology with an amino acid sequence represented by SEQ ID NO:2, SEQ ID NO:4, SEQ ID NO:6, SEQ ID NO:8 or SEQ ID NO:10 or SEQ ID NO:12 and having enhanced expression specific to RA patients (called homologous polypeptide hereinbelow).

The “functionally equivalent variant of the present invention” includes “a polypeptide comprising an amino acid sequence represented by SEQ ID NO:2, SEQ ID NO:4, SEQ ID NO:6, SEQ ID NO:8 or SEQ ID NO:10 and having enhanced expression specific to RA patients” or “a polypeptide comprising an amino acid sequence represented by SEQ ID NO:2, SEQ ID NO:4, SEQ ID NO:6, SEQ ID NO:8 or SEQ ID NO:10 in which 1 to 10, preferably 1 to 7, more preferably 1 to 5 amino acids are substituted, deleted and/or inserted and having enhanced expression specific to RA patients”.

Although the “homologous polypeptide of the present invention” is not limited as far as “a polypeptide comprising an amino acid sequence with 90% or more homology with an amino acid sequence represented by SEQ ID NO:2, SEQ ID NO:4, SEQ ID NO:6, SEQ ID NO:8 or SEQ ID NO:10 and having enhanced expression specific to RA patients”. Preferably, the homologous polypeptide of the present invention is a polypeptide comprising an amino acid sequence with preferably 95% or more homology, more preferably 98% or more homology.

In this specification, the “homology” means the value of Identities obtained by using the b12seq program (Tatiana A. Tatsusova, Thomas L. Madden, FEMS Microbiol. Lett., 174, 247-250, 1999) in the BLAST PACKAGE [sgi32 bit edition, Version 2.0.12; available from National Center for Biotechnology Information (NCBI)]. As the parameters, herein, pair wise alignment parameters are individually used, including “balstp” as “program name”, “0” as “Gap insertion Cost value”, “0” as “Gap extension Cost value”, and “BLOSUM62” as “Matrix”.

The phrase “enhanced expression specific to RA patients” means expression enhanced 2-fold or more in the synovial tissue or synovial fibroblast-like cell of RA patients, compared with non-RA patients.

The polypeptides of the present invention have been described so far. The polypeptide comprising an amino acid sequence represented by SEQ ID NO:2, SEQ ID NO:4, SEQ ID NO:6, SEQ ID NO:8 or SEQ ID NO:10, the functionally equivalent variant of the present invention and the homologous polypeptide of the present invention are generally called as “the polypeptide of the present invention” hereinafter. Further, “the polypeptide of the present invention”, a polypeptide comprising an amino acid sequence represented by SEQ ID NO:12 and having enhanced expression specific to RA patients, a polypeptide comprising an amino acid sequence represented by SEQ ID NO:12 in which 1 to 10, preferably 1 to 7, more preferably 1 to 5 amino acids are substituted, deleted and/or inserted and having enhanced expression specific to RA patients, or a polypeptide consisting of an amino acid sequence represented by SEQ ID NO:12 is generally called as “the polypeptide for the detection method of the present invention” hereinafter. In “the polypeptide for the detection method of the present invention”, a protein which is the polypeptide consisting of an amino acid sequence represented by SEQ ID NO:2 is called as “RA2 protein”; a protein which is the polypeptide consisting of an amino acid sequence represented by SEQ ID NO:4 is called as “RA3 protein”; a protein which is the polypeptide consisting of an amino acid sequence represented by SEQ ID NO:6 is called as “DGPP1L protein”; a protein which is the polypeptide consisting of an amino acid sequence represented by SEQ ID NO:8 is called as “DGPP1S protein”; a protein which is the polypeptide consisting of an amino acid sequence represented by SEQ ID NO:10 is called as “DGPP2L protein”; and a protein which is the polypeptide consisting of an amino acid sequence represented by SEQ ID NO:12 is called as “DGPP2S protein”.

The polypeptide of the present invention is preferably “a polypeptide consisting of an amino acid sequence represented by SEQ ID NO:2, SEQ ID NO:4, SEQ ID NO:6, SEQ ID NO:8 or SEQ ID NO:10”, a polypeptide comprising an amino acid sequence represented by SEQ ID NO:2, SEQ ID NO:4, SEQ ID NO:6, SEQ ID NO:8 or SEQ ID NO:10 and involving enhanced expression specific to RA patients or a polypeptide comprising an amino acid sequence represented by SEQ ID NO:2, SEQ ID NO:4, SEQ ID NO:6, SEQ ID NO:8 or SEQ ID NO:10 in which 1 to 10, preferably 1 to 7, more preferably 1 to 5 amino acids are substitutes, deleted and/or inserted and having enhanced expression specific to RA patients”, or “a polypeptide comprising an amino acid sequence with 90% or more (preferably 95% or more, more preferably 98% or more) homology with an amino acid sequence represented by SEQ ID NO:2; SEQ ID NO:4, SEQ ID NO:6, SEQ ID NO:8 or SEQ ID NO:10 and having enhanced expression specific to RA patients”. The polypeptide of the present invention is more preferably “a polypeptide consisting of an amino acid sequence represented by SEQ ID NO:2, SEQ ID NO:4, SEQ ID NO:6, SEQ ID NO:8 or SEQ ID NO:10”.

Additionally, the polynucleotide consisting of a nucleotide sequence encoding the RA2 protein of the present invention may be any of a nucleotide sequence encoding the RA2 protein represented by the amino acid sequence of SEQ ID NO:2, a functionally equivalent variant thereof or a homologous polypeptide thereof. The polynucleotide is preferably a polynucleotide consisting of a nucleotide sequence encoding the amino acid sequence of SEQ ID NO:2 and is more preferably the nucleotide sequence of SEQ ID NO:1.

The polynucleotide consisting of a nucleotide sequence encoding the RA3 protein of the present invention may be any of a nucleotide sequence encoding the RA3 protein represented by the amino acid sequence of SEQ ID NO:4, a functionally equivalent variant thereof or a homologous polypeptide thereof. The polynucleotide is preferably a polynucleotide consisting of a nucleotide sequence encoding the amino acid sequence of SEQ ID NO:4 and is more preferably the nucleotide sequence of SEQ ID NO:3.

The polynucleotide consisting of a nucleotide sequence encoding the DGPP1L protein of the present invention may be any of a nucleotide sequence encoding the DGPP1L protein represented by the amino acid sequence of SEQ ID NO:6, a functionally equivalent variant thereof or a homologous polypeptide thereof. The polynucleotide is preferably a polynucleotide consisting of a nucleotide sequence encoding the amino acid sequence of SEQ ID NO:6 and is more preferably the nucleotide sequence of SEQ ID NO:5.

The polynucleotide consisting of a nucleotide sequence encoding the DGPP1S protein of the present invention may be any of a nucleotide sequence encoding the DGPP1S protein represented by the amino acid sequence of SEQ ID NO:8, a functionally equivalent variant thereof or a homologous polypeptide thereof. The polynucleotide is preferably a polynucleotide consisting of a nucleotide sequence encoding the amino acid sequence of SEQ ID NO:8 and is more preferably the nucleotide sequence of SEQ ID NO:7.

The polynucleotide consisting of a nucleotide sequence encoding the DGPP2L protein of the present invention may be any of a nucleotide sequence encoding the DGPP2L protein represented by the amino acid sequence of SEQ ID NO:10, a functionally equivalent variant thereof or a homologous polypeptide thereof. The polynucleotide is preferably a polynucleotide consisting of a nucleotide sequence encoding the amino acid sequence of SEQ ID NO:10 and is more preferably the nucleotide sequence of SEQ ID NO:9.

The polynucleotide consisting of a nucleotide sequence encoding the DGPP2S protein of the present invention may be any of a nucleotide sequence encoding the DGPP2S protein represented by the amino acid sequence of SEQ ID NO:12, a functionally equivalent variant thereof or a homologous polypeptide thereof. The polynucleotide is preferably a polynucleotide consisting of a nucleotide sequence encoding the amino acid sequence of SEQ ID NO:12 and is more preferably the nucleotide sequence of SEQ ID NO:11.

The method for producing a polynucleotide (including the polynucleotide of the present invention) encoding the polypeptide for the detection method of the present invention and a polynucleotide specifically hybridizing with the polynucleotide of the present invention is not particularly limited and for example includes (1) the PCR method; (2) a method using routine genetic engineering technique (in other words, a method including selecting a transformant strain containing desired amino acids from transformant strains obtained by transformation with cDNA library); or (3) a chemical synthesis method. The production methods can be individually carried out as described in WO 01/34785. Herein, the “novel protein of the present invention” in the specification of the patent application should be read as the polypeptide for the detection method of the present invention (namely, the RA2 protein, the RA3 protein, the DGPP1L protein, the DGPP1S protein, the DGPP2L protein or the DGPP2S protein), while the “gene of the present invention” therein should be read as the gene encoding the polypeptide for detection in accordance with the present invention (namely, the RA2 gene, the RA3 gene, the DGPP1L gene, the DGPP1S gene, the DGPP2L gene or the DGPP2S gene). Hereinafter, the polynucleotide of the present invention is described as an example. However, the polynucleotide encoding the polypeptide for the detection method of the present invention (more specifically, a polypeptide comprising an amino acid sequence represented by SEQ ID NO:12 and having enhanced expression specific to chronic rheumatoid arthritis patients, a polypeptide comprising an amino acid sequence represented by SEQ ID NO:12 in which 1 to 10, preferably 1 to 7, more preferably 1 to 5 amino acids are substituted, deleted, and/or inserted and having enhanced expression specific to chronic rheumatoid arthritis patients, or a polypeptide consisting of an amino acid sequence represented by SEQ ID NO.12 can also be produced in the same manner.

By the method using PCR, for example, the polynucleotide of the present invention can be produced by procedures described in “Mode for Carrying out the Invention”, 1) Method for producing protein gene, a) First production method in the patent reference described above. A mRNA is extracted from cells or tissues with an ability of producing the protein of the present invention, for example from human RA patient-derived synovial membrane. Then, the mRNA is subjected to reverse-transcriptase reaction in the presence of random primer or oligo dT primer, to synthetically prepare a first cDNA chain. Using the obtained first cDNA chain and two primer types directed for a partial region of the intended gene, the cDNA is treated by polymerase chain reaction (PCR), to obtain the polynucleotide of the present invention as a whole or as a part. More specifically, the polynucleotide of the present invention can be produced for example by the method described in Example 1.

In the method using routine genetic engineering technique, for example, the polynucleotide of the present invention can be produced by the procedures described in “Mode for Carrying out the Invention”, 1) Method for producing protein gene, a) Second production method in the patent reference described above.

By the method using chemical synthesis, the polynucleotide of the present invention can be produced for example by the methods described in, “the Mode for Carrying out the Invention”, 1) Method for producing protein gene, c) Third production method and d) Fourth production method in the patent reference described above. More specifically, the polynucleotide of the present invention may be produced by binding together nucleotide fragments produced by chemical synthetic method. Additionally, each polynucleotide (oligonucleotide) may also be synthetically produced, using a DNA synthesizer (for example, Oligo 1000M DNA Synthesizer (Beckman) or 394 DNA/RNA Synthesizer (Applied Biosystems)).

The methods for producing expression vector, host cell and protein in the present invention can be produced by methods described in, “Mode for carrying out the Invention”, 2) Methods for producing the vector of the present invention, the host cell of the present invention and the recombinant protein of the present invention in the patent reference described above. The isolated polynucleotide of the present invention is integrated into an appropriate vector DNA again, to thereby transform eukaryotic or prokaryotic host cells. By additionally introducing an appropriate promoter and a sequence which relates to the phenotypic expression, the polynucleotide can be expressed in individual host cells.

The expression vector of the present invention is not particularly limited as far as it comprises the polynucleotide of the present invention, and includes such as an expression vector obtained by inserting the polynucleotide of the present invention into known expression vectors appropriately selected, depending on the host cell used.

Further, the cell of the present invention is not particularly limited as far as it is transfected with the expression vector of the present invention described above and it comprises the polynucleotide of the present invention, and includes for example cells in which the polynucleotide of the present invention is integrated in the chromosome of the host cells, or cells containing the polynucleotide of the present invention in the form of an expression vector comprising the polynucleotide. Additionally, the cell may be a cell which expresses the polypeptide of the present invention or a cell which does not expresses the polypeptide of the present invention. The cell of the present invention can be obtained by transfecting a desired host cell with the expression vector of the present invention. More specifically, for example, as described in Example 1 and Example 2, by integrating the polynucleotide of the present invention in an expression vector pcDNA3.1 for mammalian cells, an expression vector for a desired protein can be obtained, and then, by incorporating the expression vector in a human fetal kidney-derived 293T cell with the use of a commercially available transfection reagent Lipofectamine 2000, the transformant cell of the present invention can be produced.

The desired transformant cell obtained above can be cultured by routine methods. Through the culturing, the protein of the present invention is produced. As the medium for use in the culture, various medium routinely used can be selected appropriately in accordance with a selected host cell. For the 293T cell, for example, the Dulbecco's modified Eagle's minimum essential culture medium (DMEM) supplemented for example with a serum component such as fetal bovine serum (FBS) and additionally supplemented with G418 may be used.

The protein of the present invention produced in the transformant cell as described above can be separated and purified by various known separation procedures and methods using the physical properties and biochemical properties of the protein and the like.

The protein of the present invention is fused in-frame with a marker sequence and is then expressed, to enable the identification and purification of the protein and the like. As the marker sequence, for example, FLAG epitope, hexa-histidine tag, hemagglutinin tag, and mye epitope are known. By additionally inserting amino acid sequences specifically recognized by proteases, such as enterokinase, Factor Xa and thrombin in between the marker sequence and the novel protein, the marker sequence part can be cleaved and removed with these proteases.

The polynucleotide of the present invention per se or a part thereof can be used as a hybridization probe in the detection method of RA as described below and is therefore useful for detection of RA. Additionally, the polypeptide of the present invention can be used for producing an antibody which specifically recognizes the polypeptide of the present invention or as a control for detecting and assaying the expression level.

<Method for Producing the Antibody of the Invention>

Although the method for producing the antibody of the present invention is not particularly limited, it can be carried out in the same manner as described in the “Mode for Carrying out the Invention”, <Antibody binding to the polypeptide of the invention> in the patent reference described above. The antibody of the present invention, for example the polyclonal antibody or monoclonal antibody, can be obtained by direct administration of a polypeptide comprising an amino acid sequence represented by SEQ ID NO:4 and having enhanced expression specific to chronic rheumatoid arthritis patients, a polypeptide comprising an amino acid sequence represented by SEQ ID NO:4 in which 1 to several amino acids are substituted, deleted, and/or inserted, and having enhanced expression specific to chronic rheumatoid arthritis patients, or the whole or a part of a polypeptide consisting of an amino acid sequence represented by SEQ ID NO:4 to various animals. Additionally, the antibody may be obtained by a DNA vaccination method using a plasmid in which the gene encoding the polypeptide is introduced (Raz, E. et al., Proc. Natl. Acad. Sci. USA, 91, 9519-9523, 1994; Donnelly, J. J. et al., J. Infect. Dis., 173, 314-320, 1996).

Such polyclonal antibody can be produced from the serum or egg of a sensitized animal, for example sensitized rabbit, rat, goat or chicken, which is prepared by emulsifying the polypeptide or a part thereof in an appropriate adjuvant such as Freund's complete adjuvant and immunizing an animal intraperitoneally, subcutaneously or intravenously. The polyclonal antibody thus produced can be separated and purified by routine methods for protein isolation and purification. The routine methods for protein isolation and purification include for example centrifugation, dialysis, salting out with ammonium sulfate and chromatography for example with DEAE-cellulose, hydroxyapatite, and Protein A agarose.

The monoclonal antibody can be produced easily by a person skilled in the art according to the cell fusion method of Kohler and Milstein (Kohler, G. and Milstein, C., Nature, 256, 495-497, 1975).

<Polynucleotide Specifically Hybridizing to the Polynucleotide of the Invention>

A polynucleotide specifically hybridizing with a polynucleotide represented by SEQ ID NO:1, SEQ ID NO:3, SEQ ID NO:5, SEQ ID NO:7 or SEQ ID NO:9 and having at least 15 nucleotides is also included in the present invention. The phrase “specifically hybridizing” with the polynucleotide of the present invention means hybridizing with the polynucleotide of the present invention but never hybridizing with other polynucleotides under general hybridization condition, preferably under strict condition. The strict condition broadly means conditions for hybridization, such as about “5×SSPE, 5×Denhardt's solution, 0.5% SDS, 40% formamide, 200 μg/ml salmon sperm DNA, and 37° C. overnight”. More strict condition mean condition such as about “5×SSPE, 5×Denhardt's solution, 0.5% SDS, 50% formamide, 200 μg/ml salmon sperm DNA, and 42° C. overnight”. As washing condition, lax condition is the one such as about “5×SSC, 1% SDS and 42° C.”, general condition is such as about “0.5×SSC, 0.1% SDS and 42° C.”, and more strict condition is such as about “0.2×SSC, 0.1% SDS and 65° C.”. Such polynucleotide can be used as probe for the detection and isolation of the polynucleotide of the present invention or as primer for the amplification of the polynucleotide of the present invention. In case that the polynucleotide is to be used as primer, generally, the polynucleotide is of a chain length of 15 bp to 100 bp, preferably 15 bp to 40 bp. Preferable nucleotide sequences as such primer include 1) primers represented by the nucleotide sequences of SEQ ID NOS: 25 and 26, 2) primers represented by the nucleotide sequences of SEQ ID NOS: 27 and 28, 3) primers represented by the nucleotide sequences of SEQ ID NOS: 29 and 30, and 4) primers represented by the nucleotide sequences of SEQ ID NOS: 31 and 32. In case that the polynucleotide is to be used as probe, DNA comprising at least a part of the sequence of the polynucleotide or the whole (or a complimentary sequence) of the present invention and having a chain length of at least 15 bp is used.

The probe and the primer in accordance with the present invention can be used for assaying the expression level of the gene of the present invention for detection for RA diagnosis.

In accordance with the present invention, an array of oligonucleotide probes comprising the nucleotide sequence of the polynucleotide of the present invention or a fragment thereof can be constructed. The array technique is known and used for analyzing gene expression (Chee, M. et al. (1996) Science, 274, 610-613).

<RA Detection Method/Kit for RA Detection>

Since the genes which has an elevated expression levels in the synovial tissues of human RA patients was found using comparison with the synovial tissues of human osteoarthritis patients (OA), the RA disease can be detected using the expression levels thereof. So as to identify RA-specific phenomena, OA with a disorder in the synovial tissue was used as a control. Specifically, a mode including the following steps is exemplified. In other words, the mode includes

1) a step of assaying the expression level of a gene comprising a polynucleotide sequence encoding the polypeptide for the detection method of the present invention in a synovial biopsy sample of a test subject and

2) a step of comparing the expression level of the gene with the expression levels thereof in synovial biopsy samples from normal subjects or patients with non-chronic rheumatoid arthritis.

The gene expression level for the detection method of RA in the present invention includes the gene transcription into mRNA and its translation into protein. Thus, the detection method of RA in accordance with the present invention is carried out by comparison with the expression level of mRNA corresponding to the gene of the present invention or the expression level of the protein encoded by the gene.

The method for assaying the expression level of a gene comprising a polynucleotide sequence encoding the polypeptide for the detection method of the present invention in the step (1) can be carried out according to known gene analysis methods. For example, hybridization technique using nucleic acid which hybridizes with the gene of the present invention as probe, or gene amplification technique using DNA which hybridizes with a polynucleotide encoding the polypeptide for the detection method of the present invention as probe can be utilized. Specifically, nucleic acid derived from synovial cells from a test subject, for example mRNA can be used for the, assay. The mRNA amount can be assayed by the gene amplification method using a primer designed so as to specifically amplify the gene sequence of the present invention. The gene amplification method is not particularly limited and for example, PCR and nucleic acid amplification method using RNA polymerase can be used with no specific limitation. The primer for use in the detection method of RA in the present invention or the primer contained in the kit for RA detection in the present invention are not particularly limited as far as it can specifically amplify the gene sequence encoding the polypeptide for the detection method of the present invention. Such primer can be designed on the basis of the nucleotide sequence of the gene of the present invention. For the PCR amplification monitor method, is enabled by using for example a primer design software Primer Express (PE Biosystems). Additionally, a polynucleotide specifically hybridizing with a polynucleotide encoding the polypeptide for the detection method in the present invention can also be used as such primer.

RA detection using hybridization technique can be carried out, using for example Northern hybridization, dot blot method and DNA microarray method. Additionally, gene amplification technique such as RT-PCR may also be used. By RT-PCR, PCR amplification monitor (real-time PCR) method (Genome Res., 6(10), 986, 1996) is used during the course of gene amplification, to thereby make a more quantitative assay of the expression of the gene of the present invention. As the PCR amplification monitor method, for example ABI PRISM 7900 (PE Biosystems) can be used. Real-time PCR is a known method, and apparatuses and kits therefor are commercially available. Using them, the method can be carried out in a simple manner. More specifically, the method can be carried out by the method described in Example 4.

At the step (1), further, as the method for assaying the expression level of the gene comprising a polynucleotide sequence encoding the polypeptide for the detection method of the present invention, a method for detecting a protein expressed by the gene can be used. As such detection method, for example, Western blotting, immunoprecipitation method and ELISA can be used, using an antibody binding to the protein, preferably an antibody specifically binding to the protein with a cell extract solution from the synovial cell obtained from a test subject.

At the step (2), comparative method is not particularly limited as far as comparing the expression level obtained at the step (1) with the expression levels in normal subjects or non-RA patients. For example, such comparison can be carried out by the method described in Example 4.

The kit for RA detection in the present invention includes at least a forward primer and a reverse primer designed for the specific amplification of a polynucleotide encoding the polypeptide for the detection method in the present invention.

A pair of the forward and reverse primers includes for example polynucleotides specifically hybridizing with a polynucleotide encoding the polypeptide for the detection method in the present invention. Other reagents which may be contained in the kit for RA detection in the present invention include for example reagents required for PCR (for example, Taq polymerase, nucleotide substrates, buffers, etc.).

### EXAMPLES

Although the present invention is now described in more detail in the following Examples, the present invention is never limited by the Examples. Unless otherwise stated, gene manipulation experimental manuals for known methods (for example, Sambrook, J. et al., “Molecular Cloning-A Testing Manual”, Cold Spring Harbor Laboratory, N.Y., 1989) and instructions attached to reagents and the like were followed.

### Example 1

Cloning of Full-Length Open Reading Frame (ORF) and Construction of Plasmid Expressing Protein

Using a primer set shown in Table 1 (SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:18, SEQ ID NO:20 and SEQ ID NO:21, or SEQ ID NO:22 and SEQ ID NO:21), human spleen-derived CDNA (Human spleen 5′-stretch plus cDNA; Invitrogen) and DNA polymerase (Pyrobest (trade mark) DNA polymerase; Takara Shuzo Co., Ltd.), PCR was carried out at 94° C. for 2 minutes and 20 times of cycle consisting of 98° C. for 10 seconds, 60° C. for 30 seconds and 72° C. for 1 minute and 30 seconds, and followed by at 72° C. for 3 minutes. The PCR product was treated with phenol and chloroform, precipitated with ethanol and dissolved in distilled water. In the obtained DNAs, DNA amplified with the primer set of SEQ ID NO:13 and SEQ ID NO: 14 was cleaved doubly with BamHI and XhoI, while other DNAs were doubly cleaved with HindIII and XhoI. The obtained products were inserted at the BamHI-XhoI site or at the HindIII-XhoI site of the expression vector pcDNA3.1-CFL. The each obtained expression plasmid was designated as CFL-RA2, CFL-RA3, CFL-DGPP1L, CFL-DGPP1S, CFL-DGPP2L, and CDL-DFPP2S. A pcDNA3.1-CFL is a plasmid prepared by inserting a double-stranded oligoDNA consisting of SEQ ID No: 23 and SEQ ID NO:24 in the XhoI-XbaI site of pcDNA3.1(+) (Invitrogen). The plasmid gives the FLAG tag to intended protein, which is then expressed. The plasmids were sequenced by dideoxy terminator method, using AB13700 DNA sequencer (Applied Biosystems), to obtain a sequence represented by SEQ ID NO:1, SEQ ID NO:3, SEQ ID NO:5, SEQ ID NO:7, SEQ ID NO:9 or SEQ ID NO:11. The full-length ORF sequence of each of the sequences was determined. The genes were individually designated as RA2, RA3, DGPP1L, DGPP1S, DGPP2L and DGPP2S. Putative amino acid sequences were shown in SEQ ID NO:2, SEQ ID NO:4, SEQ ID NO:6, SEQ ID NO:8, SEQ ID NO:10 and SEQ ID NO:12. RA2 ORF represented by SEQ ID NO:2 encoded a novel protein consisting of 357 amino acids; RA3 ORF represented by SEQ ID NO:4 encoded a novel protein consisting of 101 amino acids; DGPP1L ORF represented by SEQ ID NO:6 encoded a novel protein consisting of 313 amino acids; DGPP1S ORF represented by SEQ ID NO:8 encoded a novel protein consisting of 264 amino acids; DGPP2L ORF represented by SEQ ID NO:10 encoded a novel protein consisting of 291 amino acids; and DGPP2S ORF represented by SEQ ID NO:12 encoded a novel protein consisting of 271 amino acids.

TABLE 1

| | Forward primer | |
|---|---|---|
| | (sequence with | Reverse primer |
| | recognition sequence | (sequence with |
| | of a restriction | recognition sequence |
| | enzyme BamHI or HindIII | of a restriction enzyme |
| | at 5′ end) | XhoI at 5′ end) |
| CFL-RA2 | SEQ ID NO: 13 | SEQ ID NO: 14 |
| CFL-RA3 | SEQ ID NO: 15 | SEQ ID NO: 16 |
| CFL-DGPP1L | SEQ ID NO: 17 | SEQ ID NO: 18 |
| CFL-DGPP1S | SEQ ID NO: 19 | SEQ ID NO: 18 |
| CFL-DGPP2L | SEQ ID NO: 20 | SEQ ID NO: 21 |
| CDL-DFPP2S | SEQ ID NO: 22 | SEQ ID NO: 2 |
| FLAG | SEQ ID NO: 23 | SEQ ID NO: 24 |

### Example 2

Expression in Animal Cell Strain

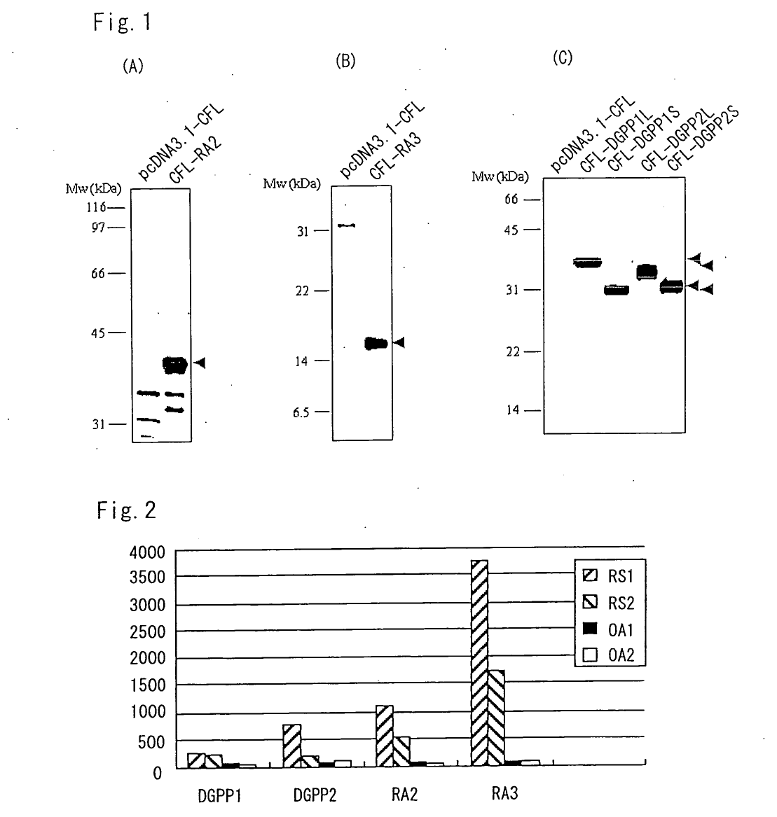

The six expression plasmids prepared in Example 1 were individually introduced in 293T cell using a transfection reagent (Lipofectamine; Gibco) according to the attached instruction. The culture medium was replaced with a serum-free culture medium 12 to 16 hours after the introduction of the plasmids, followed by additional continuous culturing for another 48 to 60 hours to collect cells A solution consisting of 50 mM Tris, pH 7.5, 0.25 M NaCl, 10% glycerol, 1% Triton X-100, 1 mM EDTA, 1 mM EGTA and 1 mM PMSF (Sigma) was added to the obtained cells to dissolve the cells, followed by centrifugation at 15,000 rpm for 15 minutes with an Eppendorf tube centrifuge machine (Tomy Digital Biology Co., Ltd.) to obtain the supernatant. The presence of an intended protein in the lysate of the introduced cell was confirmed by Western blotting using an antibody (mouse anti-FLAG monoclonal antibody M2; Sigma) against the FLAG tag added at the C terminus. In other words, the lysate described above was electrophoresed (under the reducing condition) on SDS/4% to 20 % acrylamide gel (Daiichi Pure Chemicals Co., Ltd.) and then transferred onto PVDF membrane (Millipore), using a blotting apparatus. After Block Ace (DAINIPPON PHARMACEUTICAL CO., LTD.) was added to the PVDF membrane for blocking after the transfer, a biotinylated mouse anti-FLAG monoclonal antibody and horseradish peroxidase-labeled streptoavidin (Amersham Pharmacia) were sequentially added for reaction. After reaction, the expression of the intended protein was confirmed using ECL Western blotting detection system (Amersham Pharmacia). In the lysate of the cell introduced with each of the expression plasmids of RA2, DGPP1L, DGPP1S, DGPP2L and DGPP2S, a band located around a position of each putative molecular weight (RA2: 40.1 kDa; DGPP1L: 34.3 kDa; DGPP1S: 29.5 kDa; DGPP2L: 32.3 kDa and DGPP2S: 30.4 kDa) was detected, and it can be known that the intended proteins were expressed in the each introduced cells (FIG. 1). Since RA3 has a high content of proline residue, the mobility by electrophoresis is very low and RA3 was observed at a position of a larger molecular weight than the putative molecular weight (11.0 kDa) (FIG. 1).

### Example 3

Obtaining Synovial Tissue Sample and Synovial Fibroblast-Like Cell Sample

Synovial tissues and synovial fibroblast-like cells were recovered via synovial membrane biopsy from human RA patients and human OA patients. Synovial cells were prepared according to the reference of Harigai M, et al. (J Rheumatol. May 1999; 26(5): 1035-43) while synovial fibroblast-like cells were prepared according to the reference of Zhang HG, et al. (Arthritis Rheum. May 2000; 43(5): 1094-105). G1 and G6 were samples of synovial tissues in mixture from two human RA patients. The score of various pathological findings was as follows: G1:1.5 and G6:0 in the modification of surface layer into multi layer; G1:2.5 and G6:0 in the formation of lymph follicle; G1:2.0 and G6:0.5 angiogenesis. G7 is a sample of synovial tissues in mixture from two human OA patients. The score of pathological findings was based on Harigai M., et al., Clin Immunol Immunopathol. October 1993; 69(1): 83-91 and reflects the inflammation level of synovial tissue. G1 with a high score of pathological findings is a sample with a larger inflammation level than that of G6.

R1 and R2 are synovial fibroblast-like cell samples from individual human RA patients. The scores of pathological findings are as follows: RS1:2 and RS2:0 in the modification of surface layer into multi layer; R1:2 and RS2:0 in the formation of lymph follicle; and RS1:3 and RS2:2 concerning angiogenesis. In other words, when comparing RS1 with RS2, RS1 is a sample with a larger inflammation level than that of RS2. OA1 and OA2 are synovial fibroblast-like cell samples from one human OA patient.

### Example 4

Elevation of Each Gene Expression in Synovial Tissues and Synovial Fibroblast-Like Cells

Real-time PCR with ABI PRISM 7900HT Sequence Detection System (PE Biosystems) (referred to as Prism 7900 hereinafter) was carried out using a PCR quantitative assay reagent kit SYBR Green PCR Master Mix (PE Biosystems) according to the instruction attached to the kit, to quantitatively assay the expression level of each gene in synovial tissues and synovial fibroblast-like cells. The detail is now described below. The primers for use in assaying with ABI7900 were designed with Primer Express (PE Biosystems) on the basis of the sequence information represented in SEQ ID NO:1, SEQ ID NO:3, SEQ ID NO:5, SEQ ID NO:7, SEQ ID NO:9 or SEQ ID NO:11. The primer for each gene is shown in Table 2.

TABLE 2

| | Forward primer | Reverse primer |
|---|---|---|
| RA2 | SEQ ID NO: 25 | SEQ ID NO: 26 |
| RA3 | SEQ ID NO: 27 | SEQ ID NO: 28 |
| DGPP1L, DGPP1S | SEQ ID NO: 29 | SEQ ID NO: 30 |
| DGPP2L, DGPP2S | SEQ ID NO: 31 | SEQ ID NO: 32 |
| G3DPH | SEQ ID NO: 33 | SEQ ID NO: 34 |

Total RNA (total RNA) was extracted from RA synovial tissues (G1 and G6 samples), OA synovial tissue (G7 sample), RA synovial fibroblast-like cells (RS1 and RS2 samples) and OA synovial fibroblast-like cells (OA1 and OA2 samples), using an RNA extraction reagent ISOGEN (Nippon Gene). The extraction was carried out according to the protocols attached to the reagent. The detail of the extraction method is now described below. To 100 mg of a frozen tissue, 1 ml of ISOGEN was added and homogenized, followed by standing for 5 minutes. After addition of 0.2 ml of chloroform (Kanto Chemical), the tissue was stirred and stood for 3 minutes. The tissue was centrifuged at 12,000×g at 4° C. for 15 minutes. The supernatant was recovered and 0.5 ml of 2-isopropanol (Kanto Chemical) was added thereto, followed by standing for 10 minutes. The mixture was then centrifuged at 12,000×g at 4° C. for 10 minutes. After the supernatant was removed, 1 ml of 70% ethanol was added. The resulting mixture was centrifuged at 7,500×g at 4° C. for 5 minutes. After drying in air, the product was dissolved in 50 μl of water.

The extracted total RNA was treated with DNase on a column, using a DNase processing kit (RNasey Mini kit, RNase-free DNase Set: both from Qiagen). According to the protocol attached to the kit, the treatment was carried out. The detail is described below. Water was added to the total RNA solution to become 100 μl. Subsequently, 350 ml of the buffer RLT contained in the kit was added and stirred. Then, 250 μl of ethanol was added and stirred, followed by charging in the column (RNeasy Mini spin column) included in the kit. The mixture was centrifuged at 8,000×g for 15 seconds. After adding 350 μl of the buffer RW1 contained in the kit, centrifugation at 8,000×g for 15 seconds was carried out. Further, 10 μl of the Dnase I stock solution and 70 μl of the buffer RDD contained in the kit were added, and the resulting mixture was left to stand at ambient temperature for 15 minutes. After adding 350 μl of the buffer RW1, centrifugation at 8,000×g for 15 seconds was carried out. After adding 350 μl of the buffer RW1, centrifugation at 8,000×g for 15 seconds was carried out. After adding 500 μl of the buffer RPE contained in the kit, centrifugation at 8,000×g for 15 seconds was carried out. After adding 500 μl of the buffer RPE, centrifugation at 15,000×g for 1 minute was carried out. After adding 30 gl of water, centrifugation at 8,000×g for 1 minute was carried out to obtain the total RNA solution.

By reverse transcription with total RNA as template, cDNA was prepared. Total RNA (1 μg) was mixed with 10 μl of a random hexamer (100 ng/μl) (Amersham Pharmacia Biotech), and water was added thereto to become a final volume of 48 μl. After treatment at 70° C. for 10 minutes, the resulting mixture was placed on ice. Subsequently, 32 μl of an RT reaction mixture solution was added. The mixture solution had the following composition: 5×First-Strand Buffer (250 mM Tris-HCl, pH 8.3, 375 mM KC1, 15 mM MgCl₂), 10 mM DTT, 0.5 mM DNTP, Superscript II RNase H⁻ reverse transcriptase (800 units) (all described above were from GIBCO BRL). After mixing, the resulting mixture was treated at 25° C. for 15 minutes, 42° C. for 50 minutes and 70° C. for 15 minutes.

Using the prepared CDNA as template, the following experiments were carried out. As a template for preparing a standard curve for calculating the relative expression level of an object gene among each assay samples, a dilution series of human genomic DNA (Clontech) or a dilution series of a mixture solution of each assay samples was used. In order to correct the difference in cDNA concentration in samples, G3PDH gene as an internal standard for correction was similarly assayed and analyzed. Based on the expression level of the G3PDH gene, the expression level of an intended gene was corrected and calculated. Among the expression levels of the individual genes as corrected on a G3PDH basis, the expression level of each gene in the G7 sample from OA synovial tissue or the OA1 sample from synovial fibroblast-like cell was defined as 100 and the relative expression level of each gene in each sample was calculated. The results obtained by using synovial fibroblast-like cells are shown in FIG. 2 and Table 3. In G1 which is an RA synovial tissue sample compared with G7 which is an OA synovial tissue sample, expression levels of all the genes of RA2, RA3, RA4, DGPP1, and DGPP2 were apparently and significantly elevated. Additionally, it was found that compared with G1 with a larger inflammation level, the expression levels of the individual gene described above were significantly elevated among RA synovial tissues, and that the expression levels of the individual genes are elevated in relation with the score of pathological RA findings, namely that more exacerbated RA inflammation symptoms involve more elevation of the expression levels of the individual genes. When RS1 which is an RA synovial fibroblast-like sample compared with OA1 and OA2 which are OA synovial fibroblast-like samples, expression levels of all the genes of RA2, RA3, RA4, DGPP1 and DGPP2 were apparently and significantly elevated. When the RS1 sample among the RA synovial fibroblast-like samples compared with the RS2 sample, the expression levels of the individual genes were elevated significantly. It was found that the expression levels of the individual genes were elevated in relation with the score of pathological RA findings, namely that the severity of RA inflammation symptoms has a correlation with the elevation of the expression levels of the individual genes.

These indicate that RA progress can be detected by the methods described in the Example and detection of RA diagnosis is enabled.

TABLE 3

| | DGPP1 | DGPP2 | RA2 | RA3 |
|---|---|---|---|---|
| RS1 | 273 | 762 | 4042 | 2076 |
| RS2 | 246 | 221 | 1716 | 1136 |
| OA1 | 100 | 100 | 100 | 100 |
| OA2 | 86 | 123 | 66 | 99 |

### INDUSTRIAL APPLICABILITY

It was shown that the polynucleotide of the present invention is an indicator for RA diagnosis since the elevation of the expression thereof is associated with the level of RA severity. The polynucleotide of the present invention, the antibody of the present invention and the oligonucleotide hybridizing with the polynucleotide of the present invention are useful for detection of RA diagnosis. The polypeptide of the present invention is useful for preparing the antibody of the present invention which is useful for such detection. Additionally, novel genes having enhanced expression in RA synovial cells are provided in accordance with the present invention and the novel genes can be applied to testing for RA diagnosis, by PCR using the specific primer sequence thereof.

Sequence Listing Free Text

In the numerical title <223> in the Sequence Listing below, the “Artificial Sequence” is described. Specifically, individual nucleotide sequences of SEQ ID Nos.: 13 to 24 in the Sequence Listing are primer sequences artificially synthetized.

The present invention has been described above with reference to the specific embodiments. Variations and modifications thereof obvious to persons skilled in the art are also included within the scope of the present invention.

1. (1) A polypeptide comprising an amino acid sequence represented by SEQ ID NO:2, SEQ ID NO:4, SEQ ID NO:6, SEQ ID NO:8 or SEQ ID NO:10 and having enhanced expression in chronic rheumatoid arthritis patients or
(2) a polypeptide comprising an amino acid sequence represented by SEQ ID NO:2, SEQ ID NO:4, SEQ ID NO:6, SEQ ID NO:8 or SEQ ID NO:10 in which 1 to 10 amino acids are substituted, deleted and/or inserted and having enhanced expression in chronic rheumatoid arthritis patients.

2. A polypeptide consisting of an amino acid sequence represented by SEQ ID NO:2, SEQ ID NO:4, SEQ ID NO:6, SEQ ID NO:8 or SEQ ID NO:10.

3. A polynucleotide encoding the polypeptide according to claim 1 or 2.

4. An expression vector comprising the polynucleotide according to claim 3.

5. A cell transformed with the expression vector according to claim 4.

6. A antibody which binds to a polypeptide comprising an amino acid sequence represented by SEQ ID NO:4 and having enhanced expression in chronic rheumatoid arthritis patients, a polypeptide comprising an amino acid sequence represented by SEQ ID NO:4 in which 1 to 10 amino acids are substituted, deleted and/or inserted and having enhanced expression in chronic rheumatoid arthritis patients or a polypeptide consisting of an amino acid sequence represented by SEQ ID NO:4.

7. A polynucleotide which specifically hybridizes to a polynucleotide represented by SEQ ID NO:1, SEQ ID NO:3, SEQ ID NO:5, SEQ ID NO:7 or SEQ ID NO:9 and has at least 15 nucleotides.

8. A method for detecting rheumatoid arthritis comprising
1) a step of assaying the expression level of a gene in a synovial biopsy sample of a test subject, said gene comprising
i) the nucleotide sequence according to claim 3 or
ii) a polynucleotide sequence encoding a polypeptide comprising an amino acid sequence represented by SEQ ID NO:12 and having enhanced expression in chronic rheumatoid arthritis patients, a polypeptide comprising an amino acid sequence represented by SEQ ID NO:12 in which 1 to 10 amino acids are substituted, deleted and/or inserted and having enhanced expression in chronic rheumatoid arthritis patients, or a polypeptide consisting of an amino acid sequence represented by SEQ ID NO:12; and
(2) a step of comparing the expression level of the gene with the expression levels thereof in a synovial biopsy sample in a normal subject or a patient with non-chronic rheumatoid arthritis.

9. A kit for detecting chronic rheumatoid arthritis comprising forward and reverse primers designed for enabling specific amplification of a gene represented by
1) the polynucleotide according to claim 3 or
2) a polynucleotide encoding a polypeptide comprising an amino acid sequence represented by SEQ ID NO:12 and having enhanced expression in chronic rheumatoid arthritis patients, a polypeptide comprising an amino acid sequence represented by SEQ ID NO:12 in which 1 to 10 amino acids are substituted, deleted and/or inserted and having enhanced expression in chronic rheumatoid arthritis patients, or a polypeptide consisting of an amino acid sequence represented by SEQ ID NO:12.


SEQUENCE LISTING (34 sequences)

SEQ ID NO: 1 - DNA, 1074 bp, Homo sapiens
  CDS (1)..(1071)
atgcagctcaggaatgtgtcagagcaagaactggacagcgtggccatgaagctccttcaccaagtaagcaagctgtgtgggaagtgcagccccactgacgtggacatcctgcagccctccttcaacttcctgtattggagccttcatcagaccacacccagcagtcagaaaagagctgctgcagtgctcctgagcagcacaggcctgatggagcttctggagaagatgctggccctcaccttggcaaaggcagattctcccaggactgcactcctctgctctgcctggctgctcactgcctccttctctgcccagcagcacaagggcagtttgcaggttcaccagacactctctgtggaaatggaccaagtattgaaggctctcagctttccaaagaaaaaggctgcactactctcagctgccatcttatgcttcctgcggacagccctgcgacaaagcttttcctctgccctggtagccctggtgccctcaggggcccagccactgccagccaccaaggacactgtcctagctccactgcgaatgtcgcaagtccggtccctggtcattgggctgcagaacctcctggtgcagaaggaccctctattgtcccaggcctgtgttggctgcctggaggccttgcttgactacctggatgcccggagcccagacattgctctccacgtggcctcccagccttggaatcggtttttgctgtttaccctcttggatgctggagagaattccttcctcagacctgagattttgaggctcatgaccctgtttatgcggtaccggagtagcagtgtcctctctcatgaagaggtgggtgatgttctgcaaggtgtggctttggctgacctgtctaccctctcgaacaccacactccaggccctgcatggcttcttccagcagctccagagcatgggacacctggctgaccacagcatggcccagaccctgcaggcctccttggagggccttccccctagcacctcctcaggccagccacccctgcaggacatgctctgcctgggaggggtggctgtatccctgtcccacatcagaaactga

SEQ ID NO: 2 - protein, 357 aa, Homo sapiens
MQLRNVSEQELDSVAMKLLHQVSKLCGKCSPTDVDILQPSFNFLYWSLHQTTPSSQKRAAAVLLSSTGLMELLEKMLALTLAKADSPRTALLCSAWLLTASFSAQQHKGSLQVHQTLSVEMDQVLKALSFPKKKAALLSAAILCFLRTALRQSFSSALVALVPSGAQPLPATKDTVLAPLRMSQVRSLVIGLQNLLVQKDPLLSQACVGCLEALLDYLDARSPDIALHVASQPWNRFLLFTLLDAGENSFLRPEILRLMTLFMRYRSSSVLSHEEVGDVLQGVALADLSTLSNTTLQALHGFFQQLQSMGHLADHSMAQTLQASLEGLPPSTSSGQPPLQDMLCLGGVAVSLSHIRN

SEQ ID NO: 3 - DNA, 306 bp, Homo sapiens
  CDS (1)..(303)
atgcctagaaggggaccacaacagactcgacaggatccaccggttgggcccaaggcaggaggaagggcggcgcccccaaactcccaggacgcctgcagcaccccccacgcgccgctctccgcctctggggagcatcctgccaccccccgacacacacaccccggctacatcccgccttctcacgcttggtcaggcgctctggagatgtcggagatccaggcttttcctaaagagtcaggattggaaggcggactcccaccgtttgctgagctccacatgacaacagcagacgacaggccgcactga

SEQ ID NO: 4 - protein, 101 aa, Homo sapiens
MPRRGPQQTRQDPPVGPKAGGRAAPPNSQDACSTPHAPLSASGEHPATPRHTHPGYIPPSHAWSGALEMSEIQAFPKESGLEGGLPPFAELHMTTADDRPH

SEQ ID NO: 5 - DNA, 942 bp, Homo sapiens
  CDS (1)..(939)
atgccctcggcacagccgccaggccgtcttcctggggagccgccggagcgcgggcaactgggagagggggcggtgcccagttcccggcccggccctccccgcggaggtggccacgtcagcgcagcgtcgctcggagctcgcggccggatggggaaggcggcggcggcggtggcctttggggccgaagtgggcgtgcggctcgcgctgttcgcggccttcctggtgacggagctgctccccccgttccagagactcatccagccggaggagatgtggctctaccggaacccctacgtggaggcggagtatttccccaccaagccgatgtttgttattgcatttctctctccactgtctctgatcttcctggccaaatttctcaagaaggcagacacaagagacagcagacaagcctgcctggctgccagccttgccctggctctgaatggcgtctttaccaacacaataaaactgatcgtagggaggccacgcccagatttcttctaccgctgcttccctgatgggctagcccattctgacttgatgtgtacaggggataaggacgtggtgaatgagggccgaaagagcttccccagtggacattcttcctttgcatttgctggtctggcctttgcgtccttctacctggcagggaagttacactgcttcacaccacaaggccgtgggaaatcttggaggttctgtgcctttctgtcacctctactttttgcagctgtgattgcactgtcccgcacatgtgactacaagcatcactggcaagatgtactagttggatccatgattggaatgacatttgcctatgtctgctatcggcagtattatcctcctctgactgatgcagaatgccataaaccatttcaagacaaacttgtactttccactgcacagaagcctggggattcttattgttttgatatttaa

SEQ ID NO: 6 - protein, 313 aa, Homo sapiens
MPSAQPPGRLPGEPPERGQLGEGAVPSSRPGPPRGGGHVSAASLGARGRMGKAAAAVAFGAEVGVRLALFAAFLVTELLPPFQRLIQPEEMWLYRNPYVEAEYFPTKPMFVIAFLSPLSLIFLAKFLKKADTRDSRQACLAASLALALNGVFTNTIKLIVGRPRPDFFYRCFPDGLAHSDLMCTGDKDVVNEGRKSFPSGHSSFAFAGLAFASFYLAGKLHCFTPQGRGKSWRFCAFLSPLLFAAVIALSRTCDYKHHWQDVLVGSMIGMTFAYVCYRQYYPPLTDAECHKPFQDKLVLSTAQKPGDSYCFDI

SEQ ID NO: 7 - DNA, 795 bp, Homo sapiens
  CDS (1)..(792)
atggggaaggcggcggcggcggtggcctttggggccgaagtgggcgtgcggctcgcgctgttcgcggccttcctggtgacggagctgctccccccgttccagagactcatccagccggaggagatgtggctctaccggaacccctacgtggaggcggagtatttccccaccaagccgatgtttgttattgcatttctctctccactgtctctgatcttcctggccaaatttctcaagaaggcagacacaagagacagcagacaagcctgcctggctgccagccttgccctggctctgaatggcgtctttaccaacacaataaaactgatcgtagggaggccacgcccagatttcttctaccgctgcttccctgatgggctagcccattctgacttgatgtgtacaggggataaggacgtggtgaatgagggccgaaagagcttccccagtggacattcttcctttgcatttgctggtctggcctttgcgtccttctacctggcagggaagttacactgcttcacaccacaaggccgtgggaaatcttggaggttctgtgcctttctgtcacctctactttttgcagctgtgattgcactgtcccgcacatgtgactacaagcatcactggcaagatgtactagttggatccatgattggaatgacatttgcctatgtctgctatcggcagtattatcctcctctgactgatgcagaatgccataaaccatttcaagacaaacttgtactttccactgcacagaagcctggggattcttattgttttgatatttaa

SEQ ID NO: 8 - protein, 264 aa, Homo sapiens [sequence omitted]

SEQ ID NO: 9 - DNA, 876 bp, Homo sapiens
  CDS (1)..(873)
atggctgcgggagccgcggagagcaccagctgtcgccgcgggagctgctccggccgcaccatgcgggagctggccattgagatcggggtgcgagccctgctcttcggagtcttcgtttttacagagtttttggatccgttccagagagtcatccagccagaagagatctggctctataaaaatcctttggtgcaatcagataacatacctacccgcctcatgtttgcaatttctttcctcacacccctggctgttatttgtgtggtgaaaattatccggcgaacagacaagactgaaattaaggaagccttcttagcggtgtccttggctcttgctttgaatggagtctgcacaaacactattaaattaatagtgggaagacctcgccccgatttcttttaccgctgctttccagatggagtgatgaactcggaaatgcattgcacaggtgaccccgatctggtgtccgagggccgcaaaagcttccccagcatccattcctcctttgccttttcgggccttggcttcacgacgttctacttggcgggcaagctgcactgcttcaccgagagtgggcggggaaagagctggcggctctgtgctgccatcctgcccttgtactgcgccatgatgattgccctgtcccgcatgtgcgactacaagcatcactggcaagattcctttgtgggtggagtcatcggcctcatttttgcatacatttgctacagacagcactatcctcctctggccaacacagcttgccataaaccctacgttagtctgcgagtcccagcctcactgaagaaagaggagaggcccacagctgacagcgcacccagcttgcctctggaggggatcaccgaaggcccggtatga

SEQ ID NO: 10 - protein, 291 aa, Homo sapiens
MAAGAAESTSCRRGSCSGRTMRELAIEIGVRALLFGVFVFTEFLDPFQRVIQPEEIWLYKNPLVQSDNIPTRLMFAISFLTPLAVICVVKIIRRTDKTEIKEAFLAVSLALALNGVCTNTIKLIVGRPRPDFFYRCFPDGVMNSEMHCTGDPDLVSEGRKSFPSIHSSFAFSGLGFTTFYLAGKLHCFTESGRGKSWRLCAAILPLYCAMMIALSRMCDYKHHWQDSFVGGVIGLIFAYICYRQHYPPLANTACHKPYVSLRVPASLKKEERPTADSAPSLPLEGITEGPV

SEQ ID NO: 11 - DNA, 816 bp, Homo sapiens
  CDS (1)..(813)
atgcgggagctggccattgagatcggggtgcgagccctgctcttcggagtcttcgtttttacagagtttttggatccgttccagagagtcatccagccagaagagatctggctctataaaaatcctttggtgcaatcagataacatacctacccgcctcatgtttgcaatttctttcctcacacccctggctgttatttgtgtggtgaaaattatccggcgaacagacaagactgaaattaaggaagccttcttagcggtgtccttggctcttgctttgaatggagtctgcacaaacactattaaattaatagtgggaagacctcgccccgatttcttttaccgctgctttccagatggagtgatgaactcggaaatgcattgcacaggtgaccccgatctggtgtccgagggccgcaaaagcttccccagcatccattcctcctttgccttttcgggccttggcttcacgacgttctacttggcgggcaagctgcactgcttcaccgagagtgggcggggaaagagctggcggctctgtgctgccatcctgcccttgtactgcgccatgatgattgccctgtcccgcatgtgcgactacaagcatcactggcaagattcctttgtgggtggagtcatcggcctcatttttgcatacatttgctacagacagcactatcctcctctggccaacacagcttgccataaaccctacgttagtctgcgagtcccagcctcactgaagaaagaggagaggcccacagctgacagcgcacccagcttgcctctggaggggatcaccgaaggcccggtatga

SEQ ID NO: 12 - protein, 271 aa, Homo sapiens
MRELAIEIGVRALLFGVFVFTEFLDPFQRVIQPEEIWLYKNPLVQSDNIPTRLMFAISFLTPLAVICVVKIIRRTDKTEIKEAFLAVSLALALNGVCTNTIKLIVGRPRPDFFYRCFPDGVMNSEMHCTGDPDLVSEGRKSFPSIHSSFAFSGLGFTTFYLAGKLHCFTESGRGKSWRLCAAILPLYCAMMIALSRMCDYKHHWQDSFVGGVIGLIFAYICYRQHYPPLANTACHKPYVSLRVPASLKKEERPTADSAPSLPLEGITEGPV

SEQ ID NO: 13 - DNA, 37 bp, Artificial Sequence
  chemically synthesized primer sequence
cgcgcggatccgccaccatgcagctcaggaatgtgtc

SEQ ID NO: 14 - DNA, 31 bp, Artificial Sequence
  chemically synthesized primer sequence
gcgcgctcgaggtttctgatgtgggacaggg

SEQ ID NO: 15 - DNA, 37 bp, Artificial Sequence
  chemically synthesized primer sequence
gcgcgaagcttgccaccatgcctagaaggggaccaca

SEQ ID NO: 16 - DNA, 30 bp, Artificial Sequence
  chemically synthesized primer sequence
gcgcgctcgaggtgcggcctgtcgtctgct

SEQ ID NO: 17 - DNA, 35 bp, Artificial Sequence
  chemically synthesized primer sequence
gcgcgaagcttgccaccatgccctcggcacagccg

SEQ ID NO: 18 - DNA, 35 bp, Artificial Sequence
  chemically synthesized primer sequence
gcgcgctcgagaatatcaaaacaataagaatcccc

SEQ ID NO: 19 - DNA, 35 bp, Artificial Sequence
  chemically synthesized primer sequence
gcgcgaagcttgccaccatggggaaggcggcggcg

SEQ ID NO: 20 - DNA, 35 bp, Artificial Sequence
  chemically synthesized primer sequence
gcgcgaagcttgccaccatggctgcgggagccgcg

SEQ ID NO: 21 - DNA, 30 bp, Artificial Sequence
  chemically synthesized primer sequence
gcgcgctcgagtaccgggccttcggtgatc

SEQ ID NO: 22 - DNA, 36 bp, Artificial Sequence
  chemically synthesized primer sequence
gcgcgaagcttgccaccatgcgggagctggccattg

SEQ ID NO: 23 - DNA, 31 bp, Artificial Sequence
  chemically synthesized primer sequence
tcgaggactacaaggacgacgatgacaagct

SEQ ID NO: 24 - DNA, 31 bp, Artificial Sequence
  chemically synthesized primer sequence
ctagagcttgtcatcgtcgtccttgtagtcc

SEQ ID NO: 25 - DNA, 16 bp, Homo sapiens
cacgtggcctcccagc

SEQ ID NO: 26 - DNA, 24 bp, Homo sapiens
taggttctcccatttgtcgttttt

SEQ ID NO: 27 - DNA, 20 bp, Homo sapiens
cccttgaacaacgcaggttc

SEQ ID NO: 28 - DNA, 20 bp, Homo sapiens
tttgtagggacacccacctg

SEQ ID NO: 29 - DNA, 21 bp, Homo sapiens
cttccaaggtgcaagtgagga

SEQ ID NO: 30 - DNA, 23 bp, Homo sapiens
cattggaggcagaatacagtgtg

SEQ ID NO: 31 - DNA, 20 bp, Homo sapiens
tcctgggaggatggacacta

SEQ ID NO: 32 - DNA, 21 bp, Homo sapiens
tgatgtcagggtggcagatgt

SEQ ID NO: 33 - DNA, 18 bp, Homo sapiens
gggaaggtgaaggtcgga

SEQ ID NO: 34 - DNA, 17 bp, Homo sapiens
gcagccctggtgaccag